SEQUENCE LISTING (8 sequences)

SEQ ID NO: 1 - DNA, 1128 bp, Bacillus subtilis
atgaaaaaacaacgaatgctcgtactttttaccgcactattgtttgtttttaccggatgttcacattctcctgaaacaaaagaatccccgaaagaaaaagctcagacacaaaaagtctcttcggcttctgcctctgaaaaaaaggatctgccaaacattagaattttagcgacaggaggcacgatagctggtgccgatcaatcgaaaacctcaacaactgaatataaagcaggtgttgtcggcgttgaatcactgatcgaggcagttccagaaatgaaggacattgcaaacgtcagcggcgagcagattgttaacgtcggcagcacaaatattgataataaaatattgctgaagctggcgaaacgcatcaaccacttgctcgcttcagatgatgtagacggaatcgtcgtgactcatggaacagatacattggaggaaaccgcttattttttgaatcttaccgtgaaaagtgataaaccggttgttattgtcggttcgatgagaccttccacagccatcagcgctgatgggccttctaacctgtacaatgcagtgaaagtggcaggtgcccctgaggcaaaagggaaagggacgcttgttgttcttaacgaccggattgcctcagcccgatatgtcaccaaaacaaacacaactacaacagatacatttaaatcagaagaaatgggcttcgtcggaacaattgcagatgatatctattttaataatgagattacccgtaagcatacgaaggacacggatttctcggtttctaatcttgatgagctgccgcaggttgacattatctatggataccaaaatgacggaagctacctgtttgacgctgctgtaaaagccggagcaaaggggattgtatttgccggttctgggaacgggtctttatctgatgcagccgaaaaaggggcggacagcgcagtcaaaaaaggcgttacagtggtgcgctctacccgcacgggaaatggtgtcgtcacaccaaaccaagactatgcggaaaaggacttgctggcatcgaactctttaaacccccaaaaagcacggatgttgctgatgcttgcgcttaccaaaacaaatgatcctcaaaaaatccaagcttatttcaatgagtattga

SEQ ID NO: 2 - protein, 375 aa, Bacillus subtilis
MKKQRMLVLFTALLFVFTGCSHSPETKESPKEKAQTQKVSSASASEKKDLPNIRILATGGTIAGADQSKTSTTEYKAGVVGVESLIEAVPEMKDIANVSGEQIVNVGSTNIDNKILLKLAKRINHLLASDDVDGIVVTHGTDTLEETAYFLNLTVKSDKPVVIVGSMRPSTAISADGPSNLYNAVKVAGAPEAKGKGTLVVLNDRIASARYVTKTNTTTTDTFKSEEMGFVGTIADDIYFNNEITRKHTKDTDFSVSNLDELPQVDIIYGYQNDGSYLFDAAVKAGAKGIVFAGSGNGSLSDAAEKGADSAVKKGVTVVRSTRTGNGVVTPNQDYAEKDLLASNSLNPQKARMLLMLALTKTNDPQKIQAYFNEY

SEQ ID NO: 3 - DNA, 53 bp, Bacillus subtilis
cgagctctataaaaatgaggagggaaccgaatgaaaaaacaacgaatgctcgt

SEQ ID NO: 4 - DNA, 28 bp, Bacillus subtilis
gcggccgcagaggtcattattggtccta

SEQ ID NO: 5 - DNA, 185 bp, Bacillus
ggccttaagggcctgcaatcgattgtttgagaaaagaagaagaccataaaaataccttgtctgtcatcagacagggtattttttatgctgtccagactgtccgctgtgtaaaaaaaaggaataaaggggggttgacattattttactgatatgtataatataatttgtataagaaaatggagctc

SEQ ID NO: 6 - DNA, 185 bp, Bacillus
ggccttaagggcctgcaatcgattgtttgagaaaagaagaagaccataaaaataccttgtctgtcatcagacagggtattttttatgctgtccagactgtccgctgtgtaaaaaataggaataaaggggggttgacattattttactgatatgtataatataatttgtataagaaaatggagctc

SEQ ID NO: 7 - DNA, 185 bp, Bacillus
ggccttaagggcctgcaatcgattgtttgagaaaagaagaagaccataaaaataccttgtctgtcatcagacagggtattttttatgctgtccagactgtccgctgtgtaaaaaataggaataaaggggggttgttattattttactgatatgtaaaatataatttgtataagaaaatggagctc

SEQ ID NO: 8 - DNA, 3050 bp, Bacillus
tcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgataagaaagggaggaagaaaaatgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttgactatctgcatagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaattaaattaactagaaagtaaagaagtagtgaccatctatgatagtaagcaaaggataaaaaaatgagttcataaaatgaataacatagtgttcttcaactttcgctttttgaaggtagatgaagaacactatttttattttcaaaatgaaggaagttttaaatatgtaatcatttaaagggaacaatgaaagtaggaaataagtcattatctataacaaaataacatttttatatagccagaaatgaattataatattaatcttttctaaattgacgtttttctaaacgttctatagcttcaagacgcttagaatcatcaatatttgtatacagagctgttgtttccatcgagttatgtcccatttgattcgctaatagaacaagatctttattttcgttataatgattggttgcataagtatggcgtaatttatgagggcttttcttttcatcaaaagccctcgtgtatttctctgtaagctt

(19) United States
(12) Patent Application Publication
(10) Pub. No.: US 2003/0186380 A1
(43) Pub. Date: Oct. 2, 2003

(54) Methods for producing secreted polypeptides having L-asparaginase activity

(75) Inventors: Michael D. Thomas, Davis, CA (US); Alan Sloma, Davis, CA (US)

Correspondence Address: NOVOZYMES BIOTECH, INC., 1445 DREW AVE, DAVIS, CA, 95616, US

(73) Assignee: Novozymes Biotech, Inc., Davis, CA (US)

(21) Appl. No.: 10/406,025

(22) Filed: Apr. 1, 2003

Related U.S. Application Data
(60) Provisional application No. 60/369,192, filed on Apr. 1, 2002.

(51) Int. Cl. C12P 21/02; C12N 1/21; C07H 21/04; C12N 9/64; C12N 15/74

(52) U.S. Cl. 435/69.1; 435/226; 435/320.1; 435/325; 536/23.2; 435/252.31; 435/252.33

(57) ABSTRACT

The present invention relates to recombinant methods for producing a secreted polypeptide having L-asparaginase activity, comprising (a) cultivating under conditions conducive for production of the polypeptide a host cell comprising a nucleic acid construct comprising a first nucleic acid sequence encoding a secretory signal peptide operably linked to second nucleic acid sequence encoding the polypeptide having L-asparaginase activity, wherein the signal peptide directs the polypeptide into the cell's secretory pathway; and (b) recovering the secreted polypeptide. The present invention also relates to isolated polypeptides having L-asparaginase activity and nucleic acids thereof.

### CROSS-REFERENCE TO RELATED APPLICATION

This application claims the benefit of U.S. Provisional Application No. 60/369,192, filed Apr. 1, 2002, which application is incorporated herein by reference.

### BACKGROUND OF THE INVENTION

1. Field of the Invention

The present invention relates to recombinant methods for producing secreted polypeptides having L-asparaginase activity.

2. Description of the Related Art

L-asparaginase (E.C. 3.5.1.1) catalyzes the hydrolysis of L-asparagine to L-aspartate and ammonia. L-asparaginase has been obtained from several bacterial sources.

Antitumor activity has been demonstrated with the L-asparaginase from E. coli (Hill et al., 1967, JAMA 202: 882; Capizzi et al., 1971, Ann. Intern. Med. 74: 893).

Law and Wriston, Archives of Biochemistry and Biophysics 147: 744-752 (1971), disclose the purification and properties of a non-secreted Bacillus coagulans L-asparaginase. Tyul'Panova et al., Microbiology 41: 369-374 (1972) disclose the properties of a Bacillus mesentericus 43-A L-asparaginase. Nefelova et al., Appl. Biochem. Microbiol. 14: 400-403 (1978/1979), disclose the biosynthesis of a Bacillus polymyxa L-asparaginase.

Sun and Setlow, Journal of Bacteriology 173: 3831-3845 (1971), have disclosed the cloning, nucleotide sequence, and expression of a non-secreted Bacillus subtilis L-asparaginase. Kunst et al., 1997, Nature 390: 249 disclose the complete genome sequence of Bacillus subtilis.

There is a need in the art for recombinant secreted L-asparaginases to facilitate the production and recovery of such enzymes.

It is an object of the present invention to provide secreted polypeptides having L-asparaginase activity and nucleic acids encoding such polypeptides.

### SUMMARY OF THE INVENTION

The present invention relates to recombinant methods for producing a secreted polypeptide having L-asparaginase activity, comprising (a) cultivating under conditions conducive for production of the polypeptide a host cell comprising a nucleic acid construct comprising a first nucleic acid sequence encoding a secretory signal sequence operably linked to a second nucleic acid sequence encoding the polypeptide having L-asparaginase activity; and (b) recovering the secreted polypeptide.

The present invention also relates to isolated secreted polypeptides having L-asparaginase activity selected from the group consisting of:

(a) a polypeptide having an amino acid sequence which has at least 70% identity with amino acids 24 to 375 of SEQ ID NO: 2;

(b) a polypeptide encoded by a nucleic acid sequence which hybridizes under medium stringency conditions with (i) nucleotides 70 to 1125 of SEQ ID NO: 1, (ii) a subsequence of (i) of at least 100 consecutive nucleotides, or (iii) a complementary strand of (i) or (ii); and

(c) a polypeptide fragment of (a) or (b), which has L-asparaginase activity.

The present invention also relates to isolated nucleic acid sequences encoding the secreted polypeptides and to nucleic acid constructs, vectors, and host cells comprising the nucleic acid sequences as well as methods for using the secreted polypeptides.

### BRIEF DESCRIPTION OF THE FIGURES

FIG. 1 shows the genomic DNA sequence and the deduced amino acid sequence of a Bacillus subtilis ATCC 6051A L-asparaginase (SEQ ID NOS: 1 and 2, respectively).

FIG. 2 shows a restriction map of pMDT050.

FIG. 3 shows a nucleic acid sequence containing the “consensus” amyQ promoter.

### DETAILED DESCRIPTION OF THE INVENTION

The present invention relates to recombinant methods for producing a secreted polypeptide having L-asparaginase activity, comprising (a) cultivating under conditions conducive for production of the polypeptide a host cell comprising a nucleic acid construct comprising a first nucleic acid sequence encoding a secretory signal peptide operably linked to second nucleic acid sequence encoding the polypeptide having L-asparaginase activity, wherein the signal peptide directs the polypeptide into the cell's secretory pathway; and (b) recovering the secreted polypeptide.

The methods of the present invention provide several advantages. These advantages include secretion of the L-asparaginase enabling easy recovery and purification, high expression constructs for producing the L-asparaginase in high amounts, and the use of host cells for production that have GRAS status.

The term “asparaginase activity” is defined herein as an L-asparagine amidohydrolase activity which catalyzes the hydrolysis of L-asparagine to L-aspartate and ammonia. For purposes of the present invention, L-asparaginase activity is determined according to the procedure described by da Fonseca-Wollheim, F., Bergmeyer, H. U. & Gutmann, I. (1974) in Methoden der Enzymatischen Analyse (Bergmeyer, H. U. Hrsg.) 3. Aufl., Bd. 2, S. 1850-1853, Verlag Chemie, Weinheim and (1974) in Methods of Enzymatic Analysis (Bergmeyer, H. U. ed.) 2nd ed., vol. 4, pp 1802-1806, Verlag Chemie, Weinheim/Academic Press, Inc., New York and London; and Bergmeyer, H. U. & Beutler, H. -O. (1985) in Methods of Enzymatic Analysis (Bergmeyer, H. U., ed.) 3rd ed., vol. VIII, pp. 454-461, Verlag Chemie, Weinheim, Deerfield Beach/Fla., Basel. Ammonia produced by the conversion of L-asparagine to L-aspartate by L-asparaginase is reacted with 2-oxoglutarate in the presence of glutamate dehydrogenase and reduced nicotinamide adenine dinucleotide (NADH) to produce oxidized nicotinamide adenine dinucleotide (NAD) and L-glutamate. The assay is conducted at 25° C., pH 8. One unit of L-asparaginase activity is defined as 1.0 μmole of NAD produced per minute at 25° C., pH 8.

The term “nucleic acid construct” is defined herein as a nucleic acid molecule, either single- or double-stranded, which is isolated from a naturally occurring gene or which has been modified to contain segments of nucleic acid combined and juxtaposed in a manner that would not otherwise exist in nature. The term nucleic acid construct is synonymous with the term expression cassette when the nucleic acid construct contains all the control sequences required for expression of a coding sequence. The term “coding sequence” is defined herein as a nucleic acid sequence which directly specifies the amino acid sequence of its protein product. The boundaries of a genomic coding sequence are generally determined by a ribosome binding site (prokaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

The term “operably linked” is defined herein as a configuration in which a control sequence, e.g., signal peptide sequence, is appropriately placed at a position relative to the coding sequence of the nucleic acid sequence such that the control sequence directs the expression of a polypeptide. Expression will be understood to include any step involved in the production of the polypeptide including, but not limited to, transcription, post-transcriptional modification, translation, post-translational modification, and secretion.

In the production methods of the present invention, the cells are cultivated in a nutrient medium suitable for production of the polypeptide using methods known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). Since the polypeptide is secreted into the nutrient medium, the polypeptide can be recovered directly from the medium.

The resulting secreted polypeptide may be isolated or recovered by methods known in the art. For example, the polypeptide may be recovered from the nutrient medium by conventional procedures including, but not limited to, centrifugation, filtration, extraction, spray-drying, evaporation, or precipitation.

The isolated polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989).

As defined herein, an “isolated” polypeptide is a polypeptide which is essentially free of other non-asparaginase polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

Nucleic Acid Sequences Encoding Signal Peptides

The first nucleic acid sequence encoding the secretory signal peptide is operably linked to the second nucleic acid sequence encoding the polypeptide having L-asparaginase activity. The signal peptide coding region encodes an amino acid sequence linked to the amino terminus of the polypeptide having L-asparaginase activity. The signal peptide directs the encoded polypeptide into the cell's secretory pathway.

Any nucleic acid sequence encoding a signal peptide may be used in the methods of the present invention. Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

In a preferred embodiment, the first nucleic acid sequence encoding the signal peptide comprises nucleotides 1 to 69 of SEQ ID NO: 1 which encode amino acids 1 to 23 of SEQ ID NO: 2, or a subsequence thereof that encodes a portion of the signal peptide which retains the ability to direct the encoded polypeptide into the cell's secretory pathway. In another preferred embodiment, the first nucleic acid sequence encoding the signal peptide is the sequence contained in plasmid pCR2.1-yccC which is contained in Escherichia coli NRRL B-30558.

Nucleic Acids Encoding Polypeptides Having L-Asparaginase Activity

The second nucleic acid sequence encoding the polypeptide having L-asparaginase activity may be obtained from microorganisms of any genus. For purposes of the present invention, the term “obtained from” as used herein in connection with a given source shall mean that the polypeptide encoded by the nucleic acid sequence is produced by the source or by a cell in which the nucleic acid sequence from the source has been inserted.

The nucleic acid sequence encoding a polypeptide having L-asparaginase activity may be obtained from a bacterial source. For example, the nucleic acid sequence may be a gram positive bacterial source such as a Bacillus strain, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis strain; or a Streptomyces strain, e.g., a Streptomyces lividans or Streptomyces murinus strain; or a gram negative bacterial strain, e.g., an E. coli or a Pseudomonas sp. strain.

In a preferred embodiment, the second nucleic acid sequence encodes a polypeptide having L-asparaginase activity selected from the group consisting of (a) a polypeptide having an amino acid sequence which has at least 70% identity with amino acids 24 to 375 of SEQ ID NO: 2; (b) a polypeptide which is encoded by a nucleic acid sequence which hybridizes under medium stringency conditions with (i) nucleotides 70 to 1125 of SEQ ID NO: 1, (ii) a subsequence of (i) of at least 100 consecutive nucleotides, or (iii) a complementary strand of (i) or (ii); (c) an allelic variant of (a) or (b); and (d) a fragment of (a), (b), or (c) that has L-asparaginase activity.

In a more preferred embodiment, the secreted polypeptides have an amino acid sequence which has a degree of identity to amino acids 24 to 375 of SEQ ID NO: 2 (i.e., the mature polypeptide) of at least about 70%, preferably at least about 80%, more preferably at least about 85%, even more preferably at least about 90%, most preferably at least about 95%, and even most preferably at least about 97%, which have L-asparaginase activity (hereinafter “homologous polypeptides”). The homologous polypeptides may have an amino acid sequence which differs by five amino acids, preferably by four amino acids, more preferably by three amino acids, even more preferably by two amino acids, and most preferably by one amino acid from amino acids 24 to 375 of SEQ ID NO: 2. For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty=3, windows=5, and diagonals=5.

Preferably, the polypeptide comprises amino acids 24 to 375 of SEQ ID NO: 2, or an allelic variant thereof; or a fragment thereof that has L-asparaginase activity. In another preferred embodiment, the polypeptide comprises amino acids 24 to 375 of SEQ ID NO: 2. In another preferred embodiment, the polypeptide consists of amino acids 24 to 375 of SEQ ID NO: 2 or an allelic variant thereof; or a fragment thereof that has L-asparaginase activity. In another preferred embodiment, the polypeptide consists of amino acids 24 to 375 of SEQ ID NO: 2.

A fragment of amino acids 24 to 375 of SEQ ID NO: 2 is a polypeptide having one or more amino acids deleted from the amino and/or carboxyl terminus of this amino acid sequence. Preferably, a fragment contains at least 305 amino acid residues, more preferably at least 320 amino acid residues, and most preferably at least 335 amino acid residues.

An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

In another more preferred embodiment, the polypeptide having L-asparaginase activity is a variant of the secreted polypeptide having an amino acid sequence of SEQ ID NO: 2 comprising a substitution, deletion, and/or insertion of one or more amino acids.

The amino acid sequences of the variant polypeptides may differ from amino acids 24 to 375 of SEQ ID NO: 2 by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/IIe, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/IIe, Leu/Val, Ala/Glu, and Asp/Gly as well as these in reverse.

In another more preferred embodiment, the secreted polypeptides having L-asparaginase activity are encoded by nucleic acid sequences which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with (i) nucleotides 70 to 1125 of SEQ ID NO: 1, (ii) a subsequence of (i), or (iii) a complementary strand of (i) or (ii) (J. Sambrook, E. F. Fritsch, and T. Maniatus, 1989, Molecular Cloning, A Laboratory Manual, 2d edition, Cold Spring Harbor, N.Y.). The subsequence of SEQ ID NO: 1 may be at least 100 nucleotides or preferably at least 200 nucleotides, and are preferably consecutive nucleotides. Moreover, the subsequence may encode a polypeptide fragment which has L-asparaginase activity. The polypeptides may also be allelic variants or fragments of the polypeptides that have L-asparaginase activity.

The nucleotides 70 to 1125 of SEQ ID NO: 1 or a subsequence thereof, as well as amino acids 24 to 375 of SEQ ID NO: 2 or a fragment thereof, may be used to design a nucleic acid probe to identify and clone DNA encoding polypeptides having L-asparaginase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Longer probes can also be used. Both DNA and RNA probes can be used. The probes are typically labeled for detecting the corresponding gene (for example, with ³²P, ³H, ³⁵S, biotin, or avidin). Such probes are encompassed by the present invention.

Thus, a genomic DNA or cDNA library prepared from such other organisms may be screened for DNA which hybridizes with the probes described above and which encodes a polypeptide having L-asparaginase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 1 or a subsequence thereof, the carrier material is used in a Southern blot. For purposes of the present invention, hybridization indicates that the nucleic acid sequence hybridizes to a labeled nucleic acid probe corresponding to the nucleic acid sequence shown in SEQ ID NO: 1, its complementary strand, or a subsequence thereof, under very low to very high stringency conditions. Molecules to which the nucleic acid probe hybridizes under these conditions are detected using X-ray film.

In a preferred embodiment, the nucleic acid probe is a nucleic acid sequence which encodes amino acids 24 to 375 of SEQ ID NO: 2, or a subsequence thereof. In another preferred embodiment, the nucleic acid probe is nucleotides 70 to 1125 of SEQ ID NO: 1. In another preferred embodiment, the nucleic acid probe is the nucleic acid sequence contained in plasmid pCR2.1-yccC which is contained in Escherichia coli NRRL B-30558, wherein the nucleic acid sequence encodes a polypeptide having L-asparaginase activity, i.e., amino acids 24 to 375.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a preferred embodiment, the second nucleic acid sequences are obtained from a Bacillus alkalophilus, Bacillus amyloliquefaciens, Bacillus brevis, Bacillus circulans, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis strain.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such polypeptides and the nucleic acids may be identified and obtained from other sources including microorganisms isolated from nature (e.g., soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding a polypeptide has been detected with the probe(s), the sequence may be isolated or cloned by utilizing techniques which are known to those of ordinary skill in the art (see, e.g., Sambrook et al., 1989, supra).

The techniques used to isolate or clone a nucleic acid sequence encoding a polypeptide are known in the art and include isolation from genomic DNA, preparation from cDNA, or a combination thereof. The cloning of the nucleic acid sequences from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain of Bacillus, or another or related organism and thus, for example, may be an allelic or species variant of the polypeptide encoding region of the nucleic acid sequence.

The term “isolated nucleic acid sequence” as used herein refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

In a preferred embodiment, the second nucleic acid sequence is selected from the group consisting of: (a) a nucleic acid sequence encoding a polypeptide having an amino acid sequence which has at least 65% identity with amino acids 24 to 375 of SEQ ID NO: 2; (b) a nucleic acid sequence having at least 65% homology with nucleotides 70 to 1125 of SEQ ID NO: 1; (c) a nucleic acid sequence which hybridizes under low, medium, medium-high, or high stringency conditions with (i) nucleotides 70 to 1125 of SEQ ID NO: 1, (ii) a subsequence of (i) of at least 100 consecutive nucleotides, or (iii) a complementary strand of (i) or (ii); (d) a nucleic acid sequence encoding a variant of amino acids 24 to 375 of SEQ ID NO: 2 comprising a substitution, deletion, and/or insertion of one or more amino acids; (e) an allelic variant of (a), (b), or (c); and (f) a subsequence of (a), (b), (c), or (e), wherein the subsequence encodes a polypeptide fragment which has L-asparaginase activity.

In a more preferred embodiment, the second nucleic acid sequences have a degree of homology to the nucleotides 70 to 1125 of SEQ ID NO: 1 of at least about 65%, preferably about 70%, preferably about 80%, more preferably about 90%, even more preferably about 95%, and most preferably about 97% homology, which encode an active polypeptide. For purposes of the present invention, the degree of homology between two nucleic acid sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=3, gap penalty=3, and windows=20.

In a most preferred embodiment, the second nucleic acid sequence is obtained from Bacillus subtilis strain 168, e.g., the nucleic acid sequence set forth in nucleotides 70 to 1125 of SEQ ID NO: 1. In another most preferred embodiment, the nucleic acid sequence is the sequence contained in plasmid pCR2.1-yccC, which is contained in E. coli NRRL B-30558. The methods of present invention also encompass nucleic acid sequences which encode a polypeptide having the amino acid sequence of amino acids 24 to 375 of SEQ ID NO: 2, which differ from SEQ ID NO: 1 by virtue of the degeneracy of the genetic code. The present invention also relates to subsequences of SEQ ID NO: 1 which encode fragments of amino acids 24 to 375 of SEQ ID NO: 2 that have L-asparaginase activity.

A subsequence of nucleotides 70 to 1125 of SEQ ID NO: 1 is a nucleic acid sequence encompassed by SEQ ID NO: 1 except that one or more nucleotides from the 5′ and/or 3′ end have been deleted. Preferably, a subsequence contains at least 915 nucleotides, more preferably at least 960 nucleotides, and most preferably at least 1005 nucleotides.

The second nucleic acid sequence may also comprise a mutant nucleic acid sequence comprising at least one mutation in nucleotides 70 to 1125 of SEQ ID NO: 1, in which the mutant nucleic acid sequence encodes a polypeptide which consists of amino acids 24 to 375 of SEQ ID NO: 2.

In another more preferred embodiment, the second nucleic acid sequences encoding a polypeptide having L-asparaginase activity are sequences which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with nucleotides 70 to 1125 of SEQ ID NO: 1 or its complementary strand; or allelic variants and subsequences thereof (Sambrook et al., 1989, supra), as defined herein.

Modification of the second nucleic acid sequence may be necessary for the synthesis of polypeptides substantially similar to the polypeptide having L-asparaginase activity of amino acids 24 to 375 of SEQ ID NO: 2. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variant sequence may be constructed on the basis of the nucleic acid sequence nucleotides 70 to 1125 of SEQ ID NO: 1, e.g., a subsequence thereof, and/or by introduction of nucleotide substitutions which do not give rise to another amino acid sequence of the polypeptide encoded by the nucleic acid sequence, but which correspond to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for L-asparaginase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

Nucleic Acid Constructs

The present invention also relates to nucleic acid constructs comprising a first nucleic acid sequence encoding a secretory signal peptide operably linked to a second nucleic acid sequence encoding the polypeptide having L-asparaginase activity, and further comprising one or more control sequences operably linked to the second nucleic acid sequence which direct the expression of the coding sequence in a suitable host cell under conditions compatible with the control sequences.

An isolated nucleic acid sequence encoding a polypeptide having L-asparaginase activity may be further manipulated in a variety of ways to provide for expression of the polypeptide. Manipulation of the nucleic acid sequence prior to its insertion into a vector may be desirable or necessary depending on the expression vector. The techniques for modifying nucleic acid sequences utilizing recombinant DNA methods are well known in the art.

The term “control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, promoter, ribosome binding site, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence which is recognized by a host cell for expression of the L-asparaginase encoding sequence. The promoter sequence contains transcriptional control sequences which mediate the expression of the polypeptide. The promoter may be any nucleic acid sequence which shows transcriptional activity in the host cell of choice including consensus, mutant, truncated, and hybrid promoters, and may be obtained from genes encoding extracellular or intracellular polypeptides either homologous or heterologous to the host cell.

In a preferred embodiment, the promoter sequences may be obtained from a bacterial source. In a more preferred embodiment, the promoter sequences may be obtained from a gram positive bacterium such as a Bacillus strain, e.g., Bacillus alkalophilus, Bacillus amyloliquefaciens, Bacillus brevis, Bacillus circulans, Bacillus clausll, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

Exampled of a suitable promoters for directing the transcription of the second nucleic acid sequence in the methods of the present invention are the promoters obtained from the E. coli lac operon, Bacillus clausii alkaline protease gene (aprH), Bacillus licheniformis alkaline, protease gene (subtilisin Carlsberg gene), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus thuringiensis subsp. tenebrionis CryIIIA gene (cryIIIA) or portions thereof, Streptomyces coelicolor agarase gene (dagA), and prokaryotic beta-lactamase gene (Villa-Kamaroff et al., 1978, Proceedings of the National Academy of Sciences USA 75:3727-3731). Other promoters include the spo1 bacterial phage promoter and tac promoter (DeBoer et al., 1983, Proceedings of the National Academy of Sciences USA 80:21-25). Further promoters are described in “Useful proteins from recombinant bacteria” in Scientific American, 1980, 242:74-94; and in Sambrook, Fritsch, and Maniatus, 1989, Molecular Cloning, A Laboratory Manual, 2d edition, Cold Spring Harbor, N.Y.

The promoter sequence may also be a tandem promoter. “Tandem promoter” is defined herein as two or more promoter sequences each of which is operably linked to a coding sequence and mediates the transcription of the coding sequence into mRNA. The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the host cell, e.g., Bacillus cell.

In a preferred embodiment, the tandem promoter contains at least the amyQ promoter of the Bacillus amyloliquefaciens alpha-amylase gene. In another preferred embodiment, the tandem promoter contains at least a “consensus” promoter having the sequence TTGACA for the “−35” region and TATAAT for the “−10” region. In another preferred embodiment, the tandem promoter contains at least the amyL promoter of the Bacillus licheniformis alpha-amylase gene. In another preferred embodiment, the tandem promoter contains at least the cryIIIA promoter or portions thereof (Agaisse and Lereclus, 1994, supra).

In a more preferred embodiment, the tandem promoter contains at least the amyL promoter and the cryIIIA promoter. In another more preferred embodiment, the tandem promoter contains at least the amyQ promoter and the cryIIIA promoter. In another more preferred embodiment, the tandem promoter contains at least a “consensus” promoter having the sequence TTGACA for the “−35” region and TATAAT for the “−10” region and the cryIIIA promoter. In another more preferred embodiment, the tandem promoter contains at least two copies of the amyL promoter. In another more preferred embodiment, the tandem promoter contains at least two copies of the amyQ promoter. In another more preferred embodiment, the tandem promoter contains at least two copies of a “consensus” promoter having the sequence TTGACA for the “−35” region and TATAAT for the “−10” region. In another more preferred embodiment, the tandem promoter contains at least two copies of the cryIIIA promoter.

The construction of a “consensus” promoter may be accomplished by site-directed mutagenesis to create a promoter which conforms more perfectly to the established consensus sequences for the “−10” and “−35” regions of the vegetative “sigma A-type” promoters for Bacillus subtilis (Voskuil et al., 1995, Molecular Microbiology 17: 271-279). The consensus sequence for the “−35” region is TTGACA and for the “−10” region is TATAAT. The consensus promoter may be obtained from any promoter which can function in a Bacillus host cell.

In a preferred embodiment, the “consensus” promoter is obtained from a promoter obtained from the E. coli lac operon, Streptomyces coelicolor agarase gene (dagA), Bacillus lentus alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus thuringiensis subsp. tenebrionis CryIIIA gene (cryIIIA) or portions thereof, or prokaryotic beta-lactamase gene spo1 bacterial phage promoter.

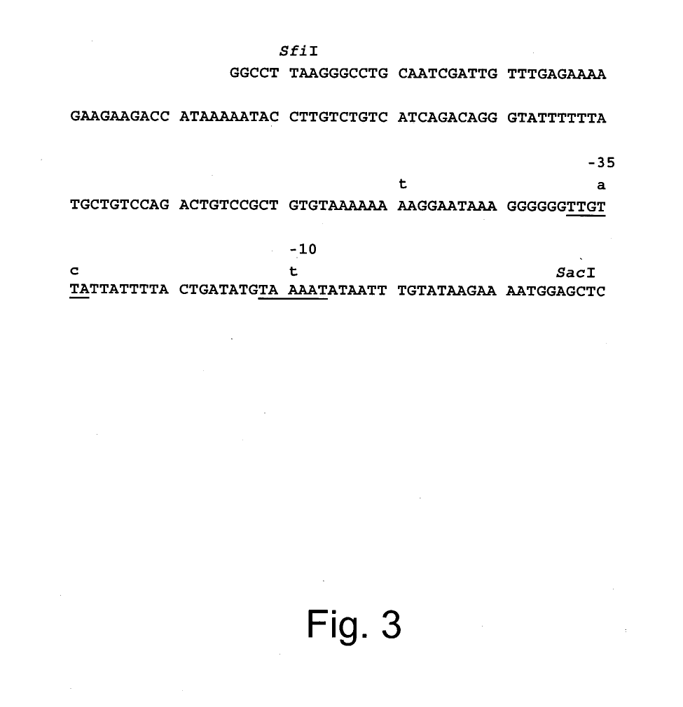

In a more preferred embodiment, the “consensus” promoter is obtained from Bacillus amyloliquefaciens alpha-amylase gene (amyQ). In a most preferred embodiment, the consensus promoter is the “consensus” amyQ promoter contained in nucleotides 1 to 185 of SEQ ID NO: 5 or SEQ ID NO: 6. In another most preferred embodiment, the consensus promoter is the short “consensus” amyQ promoter contained in nucleotides 86 to 185 of SEQ ID NO: 5 or SEQ ID NO: 6. The “consensus” amyQ promoter of SEQ ID NO: 5 contains the following mutations of the nucleic acid sequence containing the wild-type amyQ promoter (SEQ ID NO: 6): T to A and T to C in the −35 region (with respect to the transcription start site) at positions 135 and 136, respectively, and an A to T change in the −10 region at position 156 of SEQ ID NO: 7. The “consensus” amyQ promoter (SEQ ID NO: 6) further contains a T to A change at position 116 approximately 20 base pairs upstream of the −35 region as shown in FIG. 3. This change apparently had no detrimental effect on promoter function since it is well removed from the critical −10 and −35 regions.

“An mRNA processing/stabilizing sequence” is defined herein as a sequence located downstream of one or more promoter sequences and upstream of a coding sequence to which each of the one or more promoter sequences are operably linked such that all mRNAs synthesized from each promoter sequence may be processed to generate mRNA transcripts with a stabilizer sequence at the 5′ end of the transcripts. The presence of such a stabilizer sequence at the 5′ end of the mRNA transcripts increases their half-life (Agaisse and Lereclus, 1994,. supra, Hue et al., 1995, supra). The mRNA processing/stabilizing sequence is complementary to the 3′ extremity of a bacterial 16S ribosomal RNA. In a preferred embodiment, the mRNA processing/stabilizing sequence generates essentially single-size transcripts with a stabilizing sequence at the 5′ end of the transcripts.

In a more preferred embodiment, the mRNA processing/stabilizing sequence is the Bacillus thuringiensis cryIIIA mRNA processing/stabilizing sequence disclosed in WO 94/25612 and Agaisse and Lereclus, 1994, supra, or portions thereof which retain the mRNA processing/stabilizing function. In another more preferred embodiment, the mRNA processing/stabilizing sequence is the Bacillus subtilis SP82 mRNA processing/stabilizing sequence disclosed in Hue et al., 1995, supra, or portions thereof which retain the mRNA processing/stabilizing function.

When the cryIIIA promoter and its mRNA processing/stabilizing sequence are employed in the methods of the present invention, a DNA fragment containing the sequence disclosed in WO 94/25612 and Agaisse and Lereclus, 1994, supra, delineated by nucleotides −635 to −22 (SEQ ID NO: 8), or portions thereof which retain the promoter and mRNA processing/stabilizing functions, may be used. The cryIIIA promoter is delineated by nucleotides −635 to −552 while the cryIIIA mRNA processing/stabilizing sequence is contained within nucleotides −551 to −22. In a preferred embodiment, the cryIIIA mRNA processing/stabilizing sequence is contained in a fragment comprising nucleotides −568 to −22. In another preferred embodiment, the cryIIIA mRNA processing/stabilizing sequence is contained in a fragment comprising nucleotides −367 to −21. Furthermore, DNA fragments containing only the cryIIIA promoter or only the cryIIIA mRNA processing/stabilizing sequence may be prepared using methods well known in the art to construct various tandem promoter and mRNA processing/stabilizing sequence combinations. In this embodiment, the cryIIIA promoter and its mRNA processing/stabilizing sequence are preferably placed downstream of the other promoter sequence(s) constituting the tandem promoter and upstream of the coding sequence of the gene encoding a polypeptide having L-asparaginase activity. Various constructions containing a tandem promoter and the cryIIIA mRNA processing/stabilizing sequence are shown in U.S. Pat. No. 6,255,076.

In a preferred embodiment, the nucleic acid construct comprises (i) a tandem promoter in which each promoter sequence of the tandem promoter is operably linked to a single copy of a nucleic acid sequence encoding a polypeptide having L-asparaginase activity and alternatively also (ii) an mRNA processing/stabilizing sequence located downstream of the tandem promoter and upstream of the second nucleic acid sequence encoding the polypeptide.

In another preferred embodiment, the nucleic acid construct comprises (i) a “consensus” promoter operably linked to a single copy of a nucleic acid sequence encoding a polypeptide having L-asparaginase activity and alternatively also (ii) an mRNA processing/stabilizing sequence located downstream of the “consensus” promoter and upstream of the second nucleic acid sequence encoding the polypeptide. In a more preferred embodiment, the “consensus” promoter is a “consensus” amyQ promoter operably linked to a single copy of a nucleic acid sequence encoding the polypeptide. The “consensus” promoter has the sequence TTGACA for the “−35” region and TATAAT for the “−10” region.

The control sequence may also be a suitable ribosome binding site, a sequence of the mRNA recognized by the host cell to the which the ribosome binds to initiate translation. The ribosome binding site sequence is generally located between the promoter and the coding sequence. Any ribosome binding site sequence, which is functional in the host cell of choice, may be used in the present invention. For example, the ribosome binding site sequence may be obtained from the Bacillus clausii alkaline protease gene (aprH), Bacillus licheniformis alkaline protease gene (subtilisin Carlsberg gene), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus thuringiensis subsp. tenebrionis CryIIIA gene (cryIIIA) or portions thereof, Streptomyces coelicolor agarase gene (dagA), and prokaryotic beta-lactamase gene (Villa-Kamaroff et al., 1978, Proceedings of the National Academy of Sciences USA 75:3727-3731). In a preferred embodiment, the nucleic acid construct comprises the ribosome binding site sequence of the Bacillus clausii alkaline protease gene (aprH).

The control sequence may also be a suitable transcription terminator sequence, a sequence recognized by a host cell to terminate transcription. The terminator sequence is operably linked to the 3′ terminus of the nucleic acid sequence encoding the polypeptide having L-asparaginase activity. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleic acid sequence encoding the polypeptide. Any leader sequence which is functional in the host cell of choice may be used in the present invention.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE) and Bacillus subtilis neutral protease (nprT).

Where both signal peptide and propeptide regions are present at the amino terminus of a polypeptide, the propeptide region is positioned next to the amino terminus of the polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems.

The host cell may contain one or more copies of the nucleic acid construct. In a preferred embodiment, the host cell contains a single copy of the nucleic acid construct.

Expression Vectors

The present invention also relates to recombinant expression vectors comprising a first nucleic acid sequence encoding a secretory signal peptide operably linked to second nucleic acid sequence encoding the polypeptide having L-asparaginase activity, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence encoding the polypeptide having L-asparaginase activity may be expressed and secreted by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression and secretion.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, ie., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance.

The vectors of the present invention preferably contain an element(s) that permits integration of the vector into the host cell's genome or autonomous replication of the vector in the cell independent of the genome.

For integration into the host cell genome, the vector may rely on the nucleic acid sequence encoding the polypeptide having L-asparaginase activity or any other element of the vector for integration of the vector into the genome by homologous or nonhomologous recombination. Alternatively, the vector may contain additional nucleotides for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation which makes functioning temperature-sensitive in the host cell (see, e.g., Ehrlich, 1978, Proceedings of the National Academy of Sciences USA 75: 1433).

More than one copy of the nucleic acid sequence encoding the polypeptide having L-asparaginase activity may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a first nucleic acid sequence encoding a secretory signal peptide operably linked to second nucleic acid sequence encoding the polypeptide having L-asparaginase activity, which are advantageously used in the recombinant production of secreted polypeptides having L-asparaginase activity. A vector comprising the nucleic acid sequences is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any bacterial cell capable of expressing and secreting the polypeptide having L-asparaginase activity.

Useful bacterial host cells are gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus. In a more preferred embodiment, the bacterial host cell is a Bacillus subtilis strain.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Uses

The present invention also relates to methods of using the secreted polypeptides having L-asparaginase activity of the present invention.

The secreted polypeptides having L-asparaginase activity of the present invention may be used for producing L-aspartate from L-asparagine.

The secreted polypeptides of the present invention may also be useful for treatment of leukemia, e.g., acute lymphocytic leukemia (see, Asselin in Drug Resistance in Leukemia and Lymphoma III, pages 621-629, edited by Kaspers et al., Kluwer Academic/Plenum Publishers, New York, 1999).

Compositions

In a still further aspect, the present invention relates to polypeptide compositions comprising the recombinant secreted polypeptides having L-asparaginase activity. Preferably, the compositions are enriched in the secreted polypeptides having L-asparaginase activity. In the present context, the term “enriched” indicates that the L-asparaginase activity of the polypeptide composition has been increased, e.g., with an enrichment factor of 1.1.

The polypeptide composition may comprise the secreted polypeptides having L-asparaginase activity as the major or only enzymatic component, e.g., a mono-component polypeptide composition. Alternatively, the composition may comprise multiple enzymatic activities, such as an aminopeptidase, an amylase, a carbohydrase, a carboxypeptidase, a catalase, a cellulase, a chitinase, a cutinase, a cyclodextrin glycosyltransferase, a deoxyribonuclease, an esterase, an alpha-galactosidase, a beta-galactosidase, a glucoamylase, an alpha-glucosidase, a beta-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, or a xylanase. The additional enzyme(s) may be producible by means of a microorganism belonging to the genus Aspergillus, preferably Aspergillus aculeatus, Aspergillus awamori, Aspergillus niger, or Aspergillus oryzae, or Trichoderma, Humicola, preferably Humicola insolens, or Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum.

In a preferred embodiment, the composition comprises a mono-component secreted polypeptide having L-asparaginase activity and a suitable carrier. Any suitable carrier known in the art may be used.

Signal Peptide

The present invention also relates to nucleic acid constructs comprising a gene encoding a protein operably linked to a nucleic acid sequence consisting of nucleotides 1 to 69 of SEQ ID NO: 1 encoding a signal peptide consisting of amino acids 1 to 23 of SEQ ID NO: 2, wherein the gene is foreign to the nucleic acid sequences.

The present invention also relates to recombinant expression vectors and recombinant host cells comprising such nucleic acid constructs.

The present invention also relates to methods for producing a protein comprising (a) cultivating such a recombinant host cell under conditions suitable for production of the protein; and (b) recovering the protein.

The first and second nucleic acid sequences may be operably linked to foreign genes individually with other control sequences or in combination with other control sequences. Such other control sequences are described supra. As noted earlier, where both signal peptide and propeptide regions are present at the amino terminus of a protein, the propeptide region is positioned next to the amino terminus of a protein and the signal peptide region is positioned next to the amino terminus of the propeptide region.

The protein may be native or heterologous to a host cell. The term “protein” is not meant herein to refer to a specific length of the encoded product and, therefore, encompasses peptides, oligopeptides, and proteins. The term “protein” also encompasses two or more polypeptides combined to form the encoded product. The proteins also include hybrid polypeptides which comprise a combination of partial or complete polypeptide sequences obtained from at least two different proteins wherein one or more may be heterologous or native to the host cell. Proteins further include naturally occurring allelic and engineered variations of the above mentioned proteins and hybrid proteins.

Preferably, the protein is a hormone or variant thereof, enzyme, receptor or portion thereof, antibody or portion thereof, or reporter. In a more preferred embodiment, the protein is an oxidoreductase, transferase, hydrolase, lyase, isomerase, or ligase. In an even more preferred embodiment, the protein is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase or xylanase.

The gene may be obtained from any prokaryotic, eukaryotic, or other source.

The present invention is further described by the following examples which should not be construed as limiting the scope of the invention.

### EXAMPLES

Chemicals used as buffers and substrates were commercial products of at least reagent grade.

Bacterial Strains

E. coli TOP10, E. coli XL1-Blue, E. coli SURE, Bacillus subtilis A164 (ATCC 6051A), Bacillus subtilis 168 (Bacillus Stock Center, Columbus, Ohio), and Bacillus subtilis PL1801 spoIIE::Tn917 (amyE, apr, npr).

Primers and Oligos

All primers and oligos were synthesized on an Applied Biosystems Model 394 Synthesizer (Applied Biosystems, Inc., Foster City, Calif.) according to the manufacturer's instructions.

### Example 1

Isolation and Characterization of L-asparaginase Gene from Bacillus subtilis 168

Genomic DNA was isolated from Bacillus subtilis 168 using the QIAGEN bacterial genomic DNA isolation protocol (QIAGEN, Valencia, Calif.) according to the manufacturer's instructions.

Oligonucleotide primers 1 and 2 shown below were used to amplify the L-asparaginase coding region from Bacillus subtilis 168 genomic DNA by PCR. Primer 1 incorporated a SacI site and the ribosome-binding site of a Bacillus serine protease (SAVINASE™, Novo Nordisk A/S, Bagsvaerd, Denmark, hereinafter referred to as the SAVINASE™ gene) upstream of the L-asparaginase coding region, and primer 2 incorporated a NotI site downstream of the L-asparaginase coding region.

Primer 1: 5′-CGAGCTCTATAAAAATGAGGAGGGMCCGMTGAAAAAACCGAATGCTCGT-3′ (SEQ ID NO: 3)

Primer 2: 5′-GCGGCCGCAGAGGTCATTATTGGTCCTA-3′ (SEQ ID NO: 4)

The amplification reaction (50 μl) contained approximately 200 ng of Bacillus subtilis 168 genomic DNA, 0.5 μM of each primer, 200 μM each of dATP, dCTP, dGTP, and dTTP, 1×PCR buffer, 3 mM MgCl2, and 0.625 units of AmpliTaq Gold DNA polymerase (PE Applied Biosystems, Foster City, Calif.). The reaction was cycled in a RoboCycler 40 Temperature Cycler (Stratagene Cloning Systems, La Jolla, Calif.) programmed for one cycle at 95° C. for 9 minutes; 30 cycles each at 95° C. for 1 minute, 55° C. for 1 minute, and 72° C. for 2 minutes; and a final cycle at 72° C. for 3 minutes.

The PCR product was cloned using the TOPO TA Cloning Kit (Invitrogen, Carlsbad, Calif.) according to the manufacturer's instructions. Plasmid DNA was isolated from E. coli TOP10 transformants using the QIAprep 8 Plasmid Kit (QIAGEN, Valencia, Calif.) according to manufacturer's instructions. A plasmid containing the desired insert was identified by restriction analysis using enzymes EcoRI and NotI and was designated pCR2.1-yccC. The E. coli TOP10 colony containing the pCR2.1-yccC plasmid was isolated, and plasmid DNA was prepared for sequencing using a QIAGEN Plasmid Kit according to the manufacturer's instructions. E. coli SURE cells (Stratagene Cloning Systems, La Jolla, Calif.) were transformed with this plasmid, and one transformant was designated E. coli MDT50 (pCR2.1-yccC) and deposited on Feb. 8, 2002 under the terms of the Budapest Treaty with the Agricultural Research Service Patent Culture Collection, Northern Regional Research Center, 1815 University Street, Peoria, Ill., 61604, and given the accession number NRRL B-30558.

DNA sequencing was performed with an Applied Biosystems Model 377 XL Automated DNA Sequencer using dye-terminator chemistry and synthetic oligonucleotides based on the published yccC gene sequence (Kumano et al., 1997, Microbiology 143: 2775-2782). DNA sequence analysis confirmed that the sequence of the L-asparaginase gene in pCR2.1-yccC was identical to the published sequence (Kumano et al., 1997, Microbiology 143: 2775-2782).

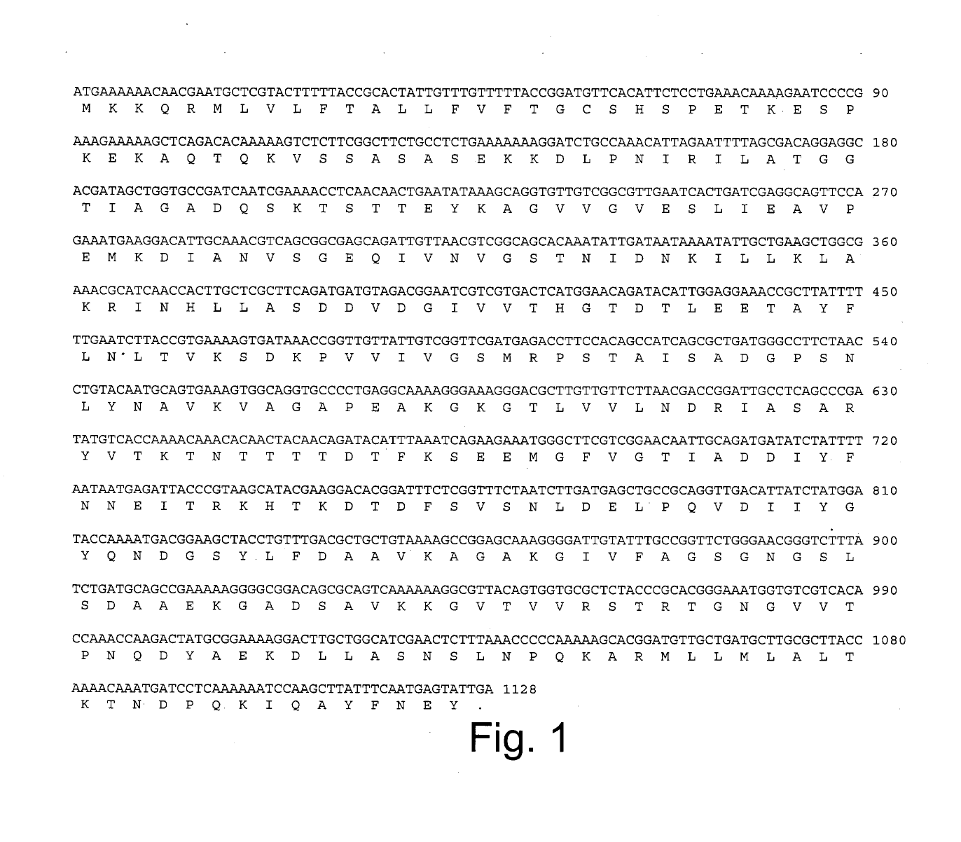

The L-asparaginase clone had an open reading frame of 1125 bp encoding a polypeptide of 375 amino acids. The nucleotide sequence (SEQ ID NO: 1) and deduced amino acid sequence (SEQ ID NO: 2) are shown in FIG. 1. Using the SignalP program (Nielsen et al., 1997, Protein Engineering 10: 1-6), a signal peptide of 23 residues was predicted corresponding to nucleotides 1 to 69.

A comparative alignment of L-asparaginase amino acid sequences was undertaken using the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty=3, windows=5, and diagonals=5.

The comparative alignment showed that the Bacillus subtilis L-asparaginase shared regions of identity of 55.2% with the L-asparaginase from Erwinia chrysanthemi (EMBL X14777) and 48.6% with L-asparaginase II of Escherichia coli (EMBL M34234).

### Example 2

Construction of pMDT050

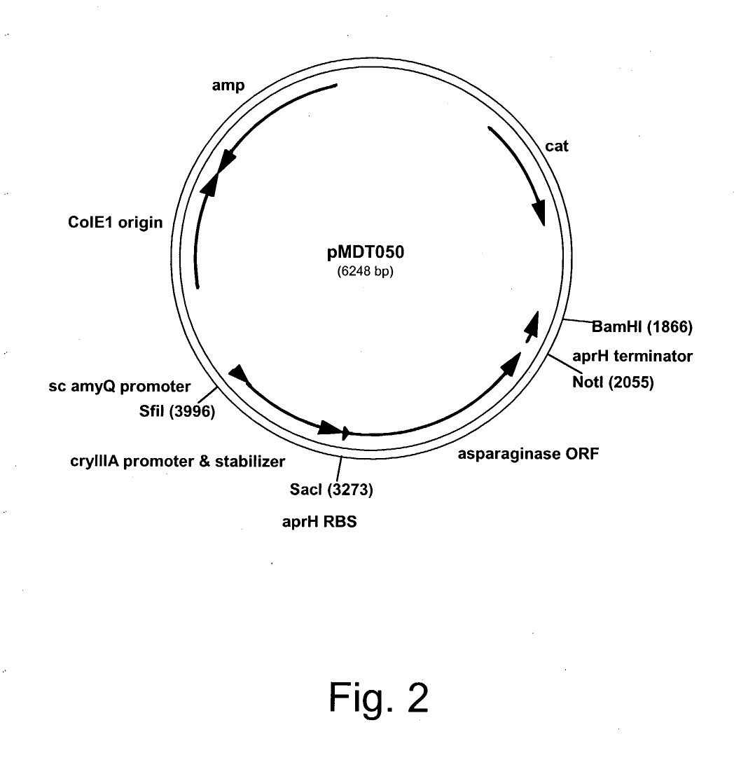

pCAsub3Δ-Pr_{“Short” consensus amyQ}/Pr_{cryIIIA}/cryIIIAstab/SAV (WO 01/14534, Example 12) was digested with SacI and NotI to remove most of the Bacillus serine protease gene coding region, and the approximately 5030 bp vector fragment was gel-purified using the QIAquick Gel Purification Kit. pCR2.1-yccC was digested with SacI and NotI, and the approximately 1220 bp L-asparaginase gene-bearing fragment was gel-purified using the QIAquick Gel Purification Kit. The gel-purified fragments were ligated using the Rapid DNA Ligation Kit. E. coli SURE cells (Stratagene Cloning Systems, La Jolla, Calif.) were transformed with the ligation mixture and ampicillin resistant transformants were selected on 2×YT plates supplemented with 100 μg of ampicillin per ml. Plasmid DNA was isolated from E. coli TOP10 transformants using the QIAprep 8 Plasmid Kit (QIAGEN, Valencia, Calif.) according to manufacturer's instructions. A plasmid containing the desired insert was identified by restriction analysis using enzyme HindIII and was designated pMDT050 (FIG. 2).

### Example 3

Construction of pMDT050 Integrant

Bacillus subtilis PL1801 spoIIE::Tn917 was transformed with pMDT050, and chloramphenicol-resistant transformants (with the pMDT050 integrated presumably at the L-asparaginase gene locus) were selected on Tryptose Blood Agar Base (TBAB) plates supplemented with 5 μg of chloramphenicol per ml. One such integrant was selected, and tandem duplications of the integrated DNA were induced by streaking an integrant on TBAB plates supplemented with progressively higher concentrations of chloramphenicol, to a maximum of 30 μg chloramphenicol per ml. This strain was designated Bacillus subtilis MDT51.

Bacillus subtilis PL1801 spoIIE::Tn917 was also transformed with pCAsub3 (WO 01/14534, Example 12) and chloramphenicol-resistant transformants were selected on TBAB plates supplemented with 5 μg of chloramphenicol per ml. One such integrant was selected, designated Bacillus subtilis MDT52, and used as a control for enzyme analyses.

### Example 4

Production of Secreted L-asparaginase

Bacillus subtilis strains MDT51 and MDT52 were grown in 50 ml of Lactobacilli MRS Broth (Difco Laboratories, Detroit, Mich.) in 250 ml shake flasks at 37° C. and 250 rpm for 24 hours. Supernatants were recovered by centrifugation at 7000 rpm for 5 minutes. Supernatant samples were run on a Novex 10-20% Tricine SDS-PAGE gel (Novex, San Diego, Calif.), and protein bands were visualized by staining with Coomassie blue. A prominent band corresponding to a protein of the expected size for mature L-asparaginase (37 kDa; amino acids 24-375) was observed in the MDT51 sample but not in the MDT52 sample.

### Example 5

Characterization of Recombinant L-asparaginase

Supernatant samples from shake flask cultures of MDT50 and MDT51 were analyzed for L-asparaginase activity. L-Asparagine was obtained from Hewlett-Packard, (Palo Alto, Calif.). Ammonia Enzymatic Bioanalysis Kit was obtained from R-Biopharm (Marshall, Mich.) (Cat. #1112732). BioSpin 6 gel filtration columns were from BioRad.

After being desalted by a BioSpin 6 column, 80 μl of MDT51 culture supernatant were mixed with 200 μl of 0.1 M asparagine (in Britton-Roberson buffer, pH 8), 20 μl water, and 300 μl of Britton-Roberson buffer, pH 8. Negative controls were run with either desaited MDT52 culture supernatant or the buffer. A positive control was run by replacing the culture supernatant with 36 μl of 0.1 M ammonium acetate. After 4 hours of incubation at 20° C., aliquots were taken for NH₃ detection. After 2 days incubation, the solution was frozen and shipped to Molecular Structure Facility of the University of California at Davis for aspartic acid detection.

Ammonia analysis was performed using an Ammonia Enzymatic Bioanalysis Kit (R-Biopharm). Samples were first diluted, if necessary, with deionized water. Then 75 μl of sample or sample dilution was combined with 625 μl of H₂O and 300 μl of Reaction Mixture #2 (containing triethanolamine buffer of pH 8, 2-oxoglutarate, NADH, stabilizers) from the kit in a quartz cuvette. The contents of the cuvette were mixed by inversion and allowed to stand at 20° C. for 5 minutes, and then the absorbance at 340 nm was measured. Then, 6 μl of Mixture #3 (containing glutamate dehydrogenase) of the kit was added and mixed. The samples were allowed to stand for 20 minutes, and the absorbance at 340 nm was measured. Calculation of NH₃ (in mg/ml) was determined using the instructions provided in the kit.

Table 1 shows the difference in NH₃ content between the MDT51 and MDT52 reactions, attributable to the deamidation of asparagine by the L-asparaginase. An apparent rate of 40 μM/h was observed.

TABLE 1
Detection of NH₃ generated from asparagine by L-asparaginase

| Broth | Asparaginase | NH₃, μg/ml | Net NH₃, μg/ml |
|---|---|---|---|
| MDT51 | + | 11.1 | 2.8 |
| MDT52 | − | 8.3 | (0) |
| Buffer | − | 0 | |
| NH₄Ac | (+) | 91.5¹ | |

¹100 μg/ml expected from the initial NH₄Ac.

Amino acid analysis was performed by the Molecular Structure Facility of the University of California at Davis. Analysis of the MDT51 reaction yielded 0.60 nmol of aspartic acid, and 6.92 of nmol asparagine per injection, corresponding to concentrations of 2.4 mM aspartic acid and 27.7 mM asparagine from a sample that contained 33 mM asparagine initially. Based on the reaction time (approximately 48 hours) and the rate estimated from the ammonia detection (approximately 40 μM/h), the sample should contain approximately 2 mM aspartic acid (generated from 33 mM asparagine by the enzyme), consistent with the observed value.

Deposit of Biological Material

The following biological material has been deposited under the terms of the Budapest Treaty with the Agricultural Research Service Patent Culture Collection, Northern Regional Research Center, 1815 University Street, Peoria, Ill., 61604, and given the following accession number:

| Deposit | Accession Number | Date of Deposit |
|---|---|---|
| E. coli MDT50 (pCR2.1-yccC) | NRRL B-30558 | Feb. 8, 2002 |

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

What is claimed is:

1. A method for producing a secreted polypeptide having L-asparaginase activity, comprising (a) cultivating under conditions conducive for production of the polypeptide a host cell comprising a nucleic acid construct comprising a first nucleic acid sequence encoding a secretory signal peptide operably linked to second nucleic acid sequence encoding the polypeptide having L-asparaginase activity, wherein the signal peptide directs the polypeptide into the cell's secretory pathway; and (b) recovering the secreted polypeptide.

2. The method of claim 1, wherein the first nucleic acid sequence encodes a secretory signal peptide comprising nucleotides 1 to 69 of SEQ ID NO: 1 which encode amino acids 1 to 23 of SEQ ID NO: 2, or a subsequence thereof that encodes a portion of the signal peptide which retains the ability to direct the encoded polypeptide into the cell's secretory pathway.

3. The method of claim 1, wherein the second nucleic acid sequence encodes a polypeptide having L-asparaginase activity selected from the group consisting of:
(a) a polypeptide having an amino acid sequence which has at least 70% identity with amino acids 24 to 375 of SEQ ID NO: 2;
(b) a polypeptide which is encoded by a nucleic acid sequence which hybridizes under medium stringency conditions with nucleotides 70 to 1125 of SEQ ID NO: 1, or a complementary strand thereof; and
(c) a fragment of (a) or (b), that has L-asparaginase activity.

4. The method of claim 3, wherein the polypeptide has an amino acid sequence which has at least 70% identity with amino acids 24 to 375 of SEQ ID NO: 2.

5. The method of claim 4, wherein the polypeptide has an amino acid sequence which has at least 80% identity with amino acids 24 to 375 of SEQ ID NO: 2.

6. The method of claim 5, wherein the polypeptide has an amino acid sequence which has at least 85% identity with amino acids 24 to 375 of SEQ ID NO: 2.

7. The method of claim 6, wherein the polypeptide has an amino acid sequence which has at least 90% identity with amino acids 24 to 375 of SEQ ID NO: 2.

8. The method of claim 7, wherein the polypeptide has an amino acid sequence which has at least 95% identity with amino acids 24 to 375 of SEQ ID NO: 2.

9. The method of claim 3, wherein the polypeptide comprises amino acids 24 to 375 of SEQ ID NO: 2.

10. The method of claim 3, wherein the polypeptide consists of amino acids 24 to 375 of SEQ ID NO: 2, or a fragment thereof that has L-asparaginase activity.

11. The method of claim 10, wherein the polypeptide consists of amino acids 24 to 375 of SEQ ID NO: 2.

12. The method of claim 3, wherein the polypeptide is encoded by a nucleic acid sequence which hybridizes under medium stringency conditions with nucleotides 70 to 1125 of SEQ ID NO: 1, or a complementary strand thereof.

13. The method of claim 3, wherein the polypeptide is encoded by a nucleic acid sequence which hybridizes under medium-high stringency conditions with nucleotides 70 to 1125 of SEQ ID NO: 1, or a complementary strand thereof.

14. The method of claim 3, wherein the polypeptide is encoded by a nucleic acid sequence which hybridizes under high stringency conditions with nucleotides 70 to 1125 of SEQ ID NO: 1, or a complementary strand thereof.

15. The method of claim 3, wherein the polypeptide is encoded by the nucleic acid sequence contained in plasmid pCR2.1-yccC which is contained in E. coli NRRL B-30558.

16. The method of claim 1, wherein the nucleic acid construct further comprises (a) a tandem promoter in which each promoter sequence of the tandem promoter is operably linked to a single copy of a nucleic acid sequence encoding a polypeptide, and optionally (b) an mRNA processing/stabilizing sequence located downstream of the tandem promoter and upstream of the second nucleic acid sequence encoding the polypeptide having L-asparaginase activity.

17. The method of claim 1, wherein the nucleic acid construct further comprises (i) a “consensus” promoter having the sequence TTGACA for the “−35” region and TATAAT for the “−10” region operably linked to a single copy of a nucleic acid sequence encoding the polypeptide and (ii) an mRNA processing/stabilizing sequence located downstream of the “consensus” promoter and upstream of the second nucleic acid sequence encoding the polypeptide having L-asparaginase activity.

18. The method of claim 17, wherein the consensus promoter is obtained from any bacterial promoter.

19. The method of claim 18, wherein the “consensus” promoter is obtained from a Bacillus promoter.

20. The method of claim 18, wherein the consensus promoter is obtained from a promoter obtained from the E. coli lac operon Streptomyces coelicolor agarase gene (dagA), Bacillus clausii alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP). Bacillus subtilis xylA and xylB genes, or Bacillus thuringiensis subsp. tenebrionis CryIIIA gene (cryIIIA) or portions thereof.

21. The method of claim 18, wherein the “consensus” promoter is obtained from the Bacillus amyloliquefaciens alpha-amylase gene (amyQ).

22. The method of claim 21, wherein the “consensus” amyQ promoter has the nucleic acid sequence of SEQ ID NO: 26 or SEQ ID NO: 27.

23. The method of claim 19, wherein the mRNA processing/stabilizing sequence is the cryIIIA mRNA processing/stabilizing sequence.

24. The method of claim 1, wherein the nucleic acid construct further comprises a ribosome binding site sequence heterologous to the host cell.

25. The method of claim 24, wherein the ribosome binding site sequence is obtained from the E. coli lac operon, Streptomyces coelicolor agarase gene (dagA), Bacillus clausii alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), or Bacillus licheniformis penicillinase gene (penP).

26. The method of claim 1, wherein the host cell is a Bacillus cell.

27. The method of claim 26, wherein the Bacillus cell is selected from the group consisting of Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis.

28. An isolated polypeptide having L-asparaginase activity, selected from the group consisting of:
(a) a polypeptide having an amino acid sequence which has at least 70% identity with amino acids 24 to 375 of SEQ ID NO: 2;
(b) a polypeptide which is encoded by a nucleic acid sequence which hybridizes under medium stringency conditions with nucleotides 70 to 1125 of SEQ ID NO: 1 or a complementary strand thereof; and
(c) a fragment of (a) or (b), that has L-asparaginase activity.

29. The polypeptide of claim 28, which has an amino acid sequence which has at least 70% identity with amino acids 24 to 375 of SEQ ID NO: 2.

30. The polypeptide of claim 29, which has an amino acid sequence which has at least 80% identity with amino acids 24 to 375 of SEQ ID NO: 2.

31. The polypeptide of claim 30, which has an amino acid sequence which has at least 85% identity with amino acids 24 to 375 of SEQ ID NO: 2.

32. The polypeptide of claim 31, wherein the polypeptide has an amino acid sequence which has at least 90% identity with amino acids 24 to 375 of SEQ ID NO: 2.

33. The polypeptide of claim 32, wherein the polypeptide has an amino acid sequence which has at least 95% identity with amino acids 24 to 375 of SEQ ID NO: 2.

34. The polypeptide of claim 28, wherein the polypeptide comprises amino acids 24 to 375 of SEQ ID NO: 2.

35. The polypeptide of claim 28, wherein the polypeptide consists of amino acids 24 to 375 of SEQ ID NO: 2, or a fragment thereof that has L-asparaginase activity.

36. The polypeptide of claim 35, wherein the polypeptide consists of amino acids 24 to 375 of SEQ ID NO: 2.

37. The polypeptide of claim 28, wherein the polypeptide is encoded by a nucleic acid sequence which hybridizes under medium stringency conditions with nucleotides 70 to 1125 of SEQ ID NO: 1, or a complementary strand thereof.

38. The polypeptide of claim 28, wherein the polypeptide is encoded by a nucleic acid sequence which hybridizes under medium-high stringency conditions with nucleotides 70 to 1125 of SEQ ID NO: 1, or a complementary strand thereof.

39. The polypeptide of claim 28, wherein the polypeptide is encoded by a nucleic acid sequence which hybridizes under high stringency conditions with nucleotides 70 to 1125 of SEQ ID NO: 1, or a complementary strand thereof.

40. The polypeptide of claim 28, wherein the polypeptide is encoded by the nucleic acid sequence contained in plasmid pCR2.1-yccC which is contained in E. coli NRRL B-30558.

41. An isolated nucleic acid sequence which encodes the polypeptide of claim 28.

42. A nucleic acid construct comprising the nucleic acid sequence of claim 41 operably linked to a secretory signal peptide encoding nucleic acid sequence and one or more control sequences that direct the production of the polypeptide in a suitable expression host.

43. A recombinant expression vector comprising the nucleic acid construct of claim 42.

44. A recombinant host cell comprising the nucleic acid construct of claim 42.

45. A nucleic acid construct comprising a gene encoding a protein operably linked to a nucleic acid sequence encoding a signal peptide consisting of nucleotides 1 to 69 of SEQ ID NO: 1, wherein the gene is foreign to the nucleic acid sequence.

46. A recombinant expression vector comprising the nucleic acid construct of claim 45.

47. A recombinant host cell comprising the nucleic acid construct of claim 45.

48. A method for producing a protein comprising (a) cultivating the recombinant host cell of claim 47 under conditions suitable for production of the protein; and (b) recovering the protein.
